(19) United States
(12) Patent Application Publication
(10) Pub. No.: US 2026/0242311 A1
(43) Pub. Date: Aug. 20, 2026

(54) PROMOTED MIXED OXIDES FOR REDOX OXIDATIVE DEHYDROGENATION OF ALKYL BENZENES WITH CUSTOMIZABLE HEAT OF REACTIONS

(71) Applicant: North Carolina State University, Raleigh, NC (US)

(72) Inventors: Fanxing Li, Raleigh, NC (US); Luke Michael Neal, Raleigh, NC (US); Aaron Frye, Raleigh, NC (US); Baitang Jin, Raleigh, NC (US)

(21) Appl. No.: 19/056,880

(22) Filed: Feb. 19, 2025

(51) Int. Cl. C07C 5/32; B01J 8/04

(52) U.S. Cl. CPC C07C 5/324; B01J 8/0492; B01J 8/0496; B01J 2208/00522; C07C 2523/78

(57) ABSTRACT

In one aspect, the disclosure relates to systems and methods for converting alkyl benzene feedstocks to alkenyl benzenes. The methods make use of both dehydrogenation and selective hydrogen combustion catalysts and may not require external steam injection. The dehydrogenation catalysts and selective hydrogen combustion catalysts can be promoted in order to increase conversion to desired products and reduce formation of unwanted side products such as coke.

### STATEMENT REGARDING FEDERALLY SPONSORED RESEARCH OR DEVELOPMENT

This invention was made with government support under grant number DE-AR0001579 awarded by the Advanced Research Projects Agency-Energy (DOE/ARPA-E). The government has certain rights in the invention.

### BACKGROUND

The disadvantages typical to industrial alkenyl benzene production from catalytic dehydrogenation of their alkyl benzene counterparts can be addressed using a chemical looping oxidative dehydrogenation (CL-ODH) scheme. A few representative alkyl benzene feedstocks typical to this production scheme are shown in FIG. 1. Generally speaking, CL-ODH can be applied to any alkyl benzene molecule with more than one side chain and with at least one side chain containing at least one carbon-carbon bond.

The CL-ODH scheme for alkenyl benzene production uses a multifunctional heterogeneous redox catalyst, i.e. a dual functional material or combination of materials that are redox active and can facilitate chemical reactions, which dehydrogenates alkyl benzenes to form alkenyl benzenes while simultaneously selectively combusting the hydrogen byproduct. In the context of chemical looping “redox active” refers to the ability of the catalyst to take up or donate oxygen during the respective steps of the loop, not merely facilitating transfers of electrons. Selective hydrogen combustion (SHC) allows CL-ODH to subvert the thermodynamic equilibrium conversion by removing one of the reaction products, increasing single-pass yields, while also providing heat to the endothermic reaction which enables autothermal reactor operation. Following the DH and SHC catalytic steps, the redox catalyst is then re-oxidized with air in a separate step. As a result, gaseous oxygen cofeeding, as employed by the UOP SMART process, is not necessary for CL-ODH as it is for conventional ODH processes. The gaseous oxygen cofeed leads to potential safety issues and hence requires stringent process safety design considerations. Additionally, gaseous oxygen cofeed for ODH can result in a reduced selectivity due to the high oxidizing activity of gaseous oxygen. CL-ODH by contrast supplies lattice oxygen using a novel mixed metal oxide promoted with a selectivity-improving salt layer, making the process safer and more selective. CL-ODH performance can be unexpectedly and exceptionally enhanced by these additions to achieve yields beyond state-of-the-art industrial processes with reduced energy consumption.

Alkyl benzenes are produced primarily from petrochemical processes where benzene, or other aromatic compounds, are alkylated with light alkanes. The resulting alkyl benzene is subsequently catalytically dehydrogenated to form the desired alkenyl benzene using a potassium-promoted iron oxide catalyst. Alkenyl benzenes are an important precursor for a wide range of high-demand polymers. The commercial process is operated at ~625° C. A total reactor (absolute) pressure of 20-50 kPa is typical used for the process. An alkyl benzene partial pressure of 2-5 kPa, with the balance being steam, is also employed. Steam is typically added on a steam-to-feed ratio (mass basis) in a range of 1-10 g steam/g feed. It should be noted the general trend that higher molecular weight alkyl benzene feedstocks require higher steam consumptions in the industrial process, sometimes with >10 steam/feed ratios for heavier feed. Due to the high reaction temperature, endothermic reaction and use of significant steam dilution, this process is in the top 15 most energy intensive processes as identified by the USDOE Chemical Bandwidth Report.

Despite advances in alkyl-to-alkenyl benzene research, there is still a scarcity of conversion methods that have high yields and minimize side products as well as lower energy demands compared to conventional methods. These needs and other needs are satisfied by the present disclosure.

### SUMMARY

In accordance with the purpose(s) of the present disclosure, as embodied and broadly described herein, the disclosure, in one aspect, relates to systems and methods for converting alkyl benzene feedstocks to alkenyl benzenes. The methods make use of both dehydrogenation and selective hydrogen combustion catalysts and may not require external steam injection. The dehydrogenation catalysts and selective hydrogen combustion catalysts can be promoted in order to increase conversion to desired products and reduce formation of unwanted side products such as coke.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 shows ethylbenzene and examples of heavier alkyl benzene feedstocks.

FIG. 2 shows CL-ODH catalyst performance with and without steam co-injection.

FIG. 3 shows a conceptual heat-map illustration according to one embodiment of the present disclosure.

FIG. 4 shows a reaction scheme of ethylbenzene CL-ODH with a cyclic redox scheme.

FIG. 5 shows a CATOFIN-type reactor schematic useful according to one embodiment of the present disclosure.

FIGS. 6A-6D show performance of sequential bed catalysts.

FIG. 7 summarizes the findings of FIGS. 6A-6D.

FIG. 8 shows composite catalyst bed performance with and without steam co-injection.

FIG. 9 shows an optimized styrene yield obtained with ethylbenzene CL-ODH using a multifunctional redox catalyst.

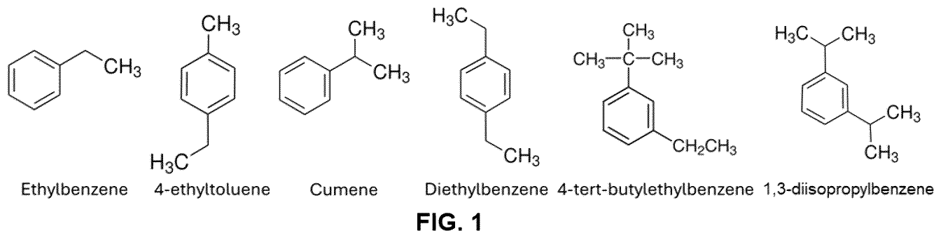

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

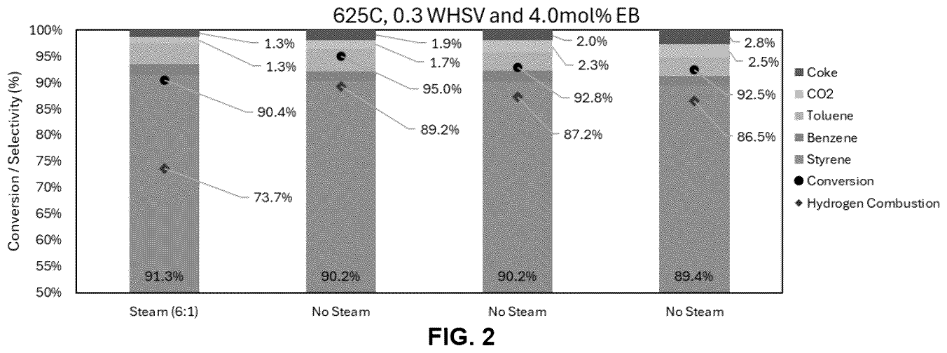

In one aspect, to improve the yield of the equilibrium-limited alkyl- to alkenyl benzene catalytic dehydrogenation reaction, hydrogen can be selectively combusted by use of a reducible mixed metal oxide (generically ABO₃, where A is typically an alkali earth metal and B is typically a transition metal). In a further aspect, CL-ODH experimental results have shown that a lower steam to feed ratio is feasible with ranges more typical of 0-5 g steam/g feed, or more preferably 0-1 g steam/g feed. In one aspect, and without wishing to be bound by theory, the unexpectedly excellent performance enhancement when no steam is injected, demonstrated in FIG. 2, is due to in-situ steam generation from the novel selective hydrogen combustion feature of the CL-ODH process. This effect is further enhanced when combined with lower overall steam partial pressure and on-purpose design of tailored mixed metal oxides for heat release and hydrogen combustion selectivity properties.

In an aspect, typical metal oxide requires a significant amount of energy to reduce, leading to an endotherm when reacting with hydrogen. Disclosed herein are the mixed metal oxides with net exothermicity of hydrogen combustion during the reduction step (energy released from oxidation of hydrogen plus energy required to release oxygen from the mixed metal oxide lattice). This exotherm may make the reduction step significantly less endothermic, or exothermic, enabling self-heating, autothermal operation for the otherwise endothermic dehydrogenation process.

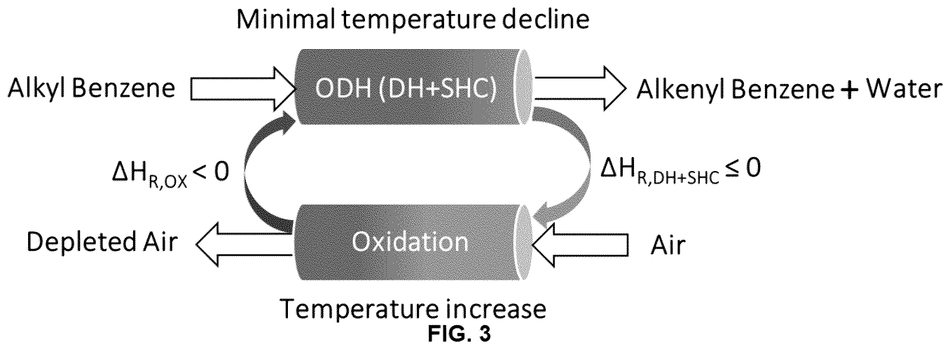

In one aspect, typically, mixed metal oxides alone unselectively combust a portion of the desirable carbonaceous reactants/products (the alkyl benzene, alkenyl benzene, or other aromatic byproducts). Also disclosed herein is the use of a novel promoter that modifies the surface catalytic properties of the mixed metal oxide, allowing for more selective oxygen transfer to primarily combust hydrogen. In one aspect, the combination of the two reactions, as shown in FIG. 3, allows the overall heat of reaction for selective hydrogen combustion to be tuned to a desirable range (<0 kJ/mol). In turn, ΔH_{R,DH+SHC} and ΔH_{R,OX}, shown in FIG. 3, may be calculated by those knowledgeable in the art.

In still another aspect, the heat of reaction from SHC may be utilized, in combination with a desirable endothermic reaction, in order to facilitate autothermal reactor operation. In one aspect, a self-heating autothermal reactor for the dehydrogenation step driven by mixed oxides would allow for higher single-pass conversions, since the heat would increase the reactor temperature and accelerate the dehydrogenation reaction, while removing the equilibrium limitation. In a further aspect, this lowers the energy conversion to produce alkenyl benzenes. It is noted that such an effect is rather unexpected given that typical mixed oxide driven CL-ODH reaction will be quite endothermic in the dehydrogenation step.

### Method for Converting an Alkyl Benzene to an Alkenyl Benzene

In one aspect, disclosed herein is a method for converting at least one alkyl benzene feedstock to an alkenyl benzene, the method including at least the steps of:
(a) contacting the at least one alkyl benzene feedstock with a dehydrogenation catalyst and a selective hydrogen combustion catalyst to produce the alkenyl benzene and selectively combust a hydrogen byproduct, wherein the selective hydrogen combustion catalyst releases at least 30 kJ of thermal energy per mole of hydrogen combusted into steam;
(b) oxidizing the dehydrogenation catalyst and the selective hydrogen combustion catalyst; and repeating steps (a) and (b) one or more additional times.

In some aspects, no external steam is injected. In another aspect, production of water in step (a) causes in situ steam generation. In one aspect, in the disclosed method, a combination of step (a) and step (b) has a heat of reaction of less than 100 kJ/mol alkyl benzene converted. In an aspect, oxidizing the dehydrogenation catalyst and the selective hydrogen combustion catalyst includes the step of exposing the dehydrogenation catalyst and the selective hydrogen combustion catalyst to air. In some aspects, no external steam is injected.

In one aspect, the dehydrogenation catalyst and the selective hydrogen combustion catalyst are arranged in a fixed bed, which can be arranged in one of the following manners alone or in combination: (i) sequentially arranged alternating catalyst beds separately including the dehydrogenation catalyst and the selective hydrogen combustion catalyst, wherein the alternating catalyst beds are separated by a physical distance; (ii) a composite bed wherein the dehydrogenation catalyst and the selective hydrogen combustion catalyst are physically in contact in the composite bed; or (iii) stacked alternating catalyst beds including the dehydrogenation catalyst and the selective hydrogen combustion catalyst, wherein the alternating catalyst beds are in physical contact with one or more adjacent catalyst beds.

In one aspect, the fixed bed includes arrangement (i) and the feedstock is subjected to steam purges between step (a) and step (b). In some aspects, the entire reactor including the fixed bed is purged with steam. In an alternative aspect, the fixed bed includes arrangement (ii) and the dehydrogenation catalyst and the selective hydrogen combustion catalyst are present in the form of admixed particles, pellets, or a monolith. In yet another aspect, the fixed bed includes arrangement (ii) and the dehydrogenation catalyst is present as a shell around a selective hydrogen combustion catalyst core. In a further aspect, the fixed bed includes arrangement (i) or (iii) and the fixed bed includes from 2 to 20 alternating catalyst beds. In any of these aspects, the fixed bed can include one or more inert support beds, wherein the one or more inert support beds are configured to ensure even heating of the alkyl benzene feedstock.

In one aspect, the dehydrogenation catalyst can be KFeO₂ or can be selected from potassium-promoted iron oxide with one or more additional promoters including, but not limited to, Ce, Ga, Ti, Mg, Mn, Cs, Na, Li, Mo, W, and their oxides and salts. In an aspect, the oxide can be TiO₂, Ga₂O₃, CeO₂, MgO, or any combination thereof. In one aspect, the dehydrogenation catalyst can be Ga₂O₃:TiO₂:CeO₂:MgO:KFeO₂ in a ratio of 2:2:2:1:13. In an aspect, the promoter includes a transition metal cation, an alkaline earth metal, or a lanthanide cation, or any combination thereof in an amount of from about 0% to about 10% by weight, or from about 0% to about 1% by weight, about 0.1% to about 5% by weight, about 0.001% to about 2% by weight, or about 1, 2, 3, 4, 5, 6, 7, 8, 9, or about 10% by weight Other active dehydrogenation catalysts known to those skilled in the art are also contemplated and should be considered disclosed. In another aspect, the selective hydrogen combustion catalyst can be selected from any redox active oxides, particularly those containing Fe, Mn, Ce, Cu, Ni, Mo, Co, W, and combinations thereof. In an aspect, the redox active elements are integrated, along with other cations from the periodic table, into a mixed oxide structure including but not limited to perovskite, spinel, Ruddlesden-Popper, Brownmillerite, bixbyite, rock salt, fluorite, hexaaluminate, or any combination thereof. In yet another aspect, the dehydrogenation catalyst, the selective hydrogen combustion catalyst, or both can include a promoter.

In one aspect, the promoter for the selective hydrogen combustion catalyst can be an alkali metal salt and can optionally include a promoter. Further in this aspect, the promoter cation can be selected from Li, Na, K, or any combination thereof. In one aspect, the salt can be a molybdate salt, a vanadate salt, a tungstate salt, or any combination thereof. In one aspect, the selective hydrogen combustion catalyst can be selected from LaSrFeO₃, Mg₆MnO₈, (Fe_{0.2}Mn_{0.8})₂O₃, LaCaMnO₃, CaMnTiFeO₃.

In one aspect, the use of the promoted SHC causes the combustion of hydrogen to be net exothermic (combined reduction of the SHC and oxidation of H₂). In another the dehydrogenation step will have a net heat of reaction <100 kJ/mol alky benzene reacted vs a >20% reduction in the theoretical exotherm, facilitating the reactor to be operated without external heat through the use of the sensible heat of the reactor bed (including catalyst and inert packing). In one aspect, the reaction endotherm will be less than <50 kJ/mol alkyl benzene reacted. Without wishing to be bound by theory, this can be beneficial as it limits the need for temperature swings >50° C. between steps, greatly reducing the mechanical stress on the catalyst and reactor and making control of the system easier. In yet another aspect, the net reduction step will be exothermic. In a further aspect, energy released in the exothermic step can be harnessed for subsequent use.

In one aspect, the alkyl benzene feedstock includes one or more side chains, wherein at least one side chain has a carbon-carbon bond. In another aspect, the alkyl benzene feedstock can be selected from ethylbenzene, 4-ethyltoluene, cumene, diethylbenzene, 4-tertbutylethylbenzene, 1,3-diisopropylbenzene, or any combination thereof. In another aspect, the alkyl benzene feedstock includes a gaseous feedstock comprising from about 1 mol % to about 100 mol %, from about 1 mol % to about 50 mol %, or from about 1 mol % to about 100 mol % of at least one alkyl benzene. In a further aspect, the gaseous feedstock can include from about 0 g to about 5 g steam per gram of feedstock.

In any of these aspects, the method suppresses coke formation. In a further aspect, the method is performed at a temperature of from about 500° C. to about 700° C., or at from about 550° C. to about 650° C., or at about 500, 550, 600, 650, or 700° C. In one aspect, at least about 80% of the alkyl benzene feedstock is converted to alkenyl benzene, or at least about 85%, 90%, 95%, 99%, or 100% of the alkyl benzene feedstock is converted to alkenyl benzene.

### System for Converting an Alkyl Benzene to an Alkenyl Benzene

In one aspect, disclosed herein is a system for converting at least one alkyl benzene feedstock to an alkenyl benzene, the system including at least the following components:
(a) a fixed bed for containing a dehydrogenation catalyst and a selective hydrogen combustion catalyst;
(b) a means for flowing an alkyl benzene feedstock over the fixed bed; and
(c) a means for flowing air over the fixed bed.

In a further aspect, the fixed bed can include one of the following arrangements:
(i) sequentially arranged alternating catalyst beds separately comprising the dehydrogenation catalyst and the selective hydrogen combustion catalyst, wherein the alternating catalyst beds are separated by a physical distance;
(ii) a composite bed wherein the dehydrogenation catalyst and the selective hydrogen combustion catalyst are physically in contact in the composite bed; or
(iii) stacked alternating catalyst beds comprising the dehydrogenation catalyst and the selective hydrogen combustion catalyst, wherein the alternating catalyst beds are in physical contact with one or more adjacent catalyst beds.

In one aspect, the fixed bed includes arrangement (i) and the system further includes a means for purging the feedstock with steam as the feedstock passes between the alternating catalyst beds. In another aspect, the fixed bed includes arrangement (ii) and the dehydrogenation catalyst and the selective hydrogen combustion catalyst are present in the form of admixed pellets, particles, or a monolith. In still another aspect, the fixed bed includes arrangement (ii) and the dehydrogenation catalyst is present as a shell around a selective hydrogen combustion catalyst core. In yet another aspect, the fixed bed includes arrangement (i) or (iii) and the fixed bed includes from 2 to 20 alternating catalyst beds. In any of these aspects, the fixed bed can also include one or more inert support beds, wherein the one or more inert support beds are configured to ensure even heating of the alkyl benzene feedstock, the air, or both.

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a mixed metal oxide,” “a catalyst promoter,” or “an alkyl benzene,” includes, but is not limited to, mixtures or combinations of two or more such mixed metal oxides, catalyst promoters, or alkyl benzenes, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the terms “optional” or “optionally” means that the subsequently described event or circumstance can or cannot occur, and that the description includes instances where said event or circumstance occurs and instances where it does not.

Unless otherwise specified, temperatures referred to herein are based on atmospheric pressure (i.e. one atmosphere).

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### Aspects

The present disclosure can be described in accordance with the following numbered aspects, which should not be confused with the claims.

Aspect 1. A method for converting at least one alkyl benzene feedstock to an alkenyl benzene, the method comprising:
(a) contacting the at least one alkyl benzene feedstock with a dehydrogenation catalyst and a selective hydrogen combustion catalyst to produce the alkenyl benzene and selectively combust a hydrogen byproduct, wherein the selective hydrogen combustion catalyst releases at least 30 kJ of thermal energy per mole of hydrogen combusted into steam;
(b) oxidizing the dehydrogenation catalyst and the selective hydrogen combustion catalyst; and
repeating steps (a) and (b) one or more additional times.

Aspect 2. The method of aspect 1, wherein a combination of step (a) and step (b) has a heat of reaction of less than 100 kJ/mol alkyl benzene converted.

Aspect 3. The method of aspect 1, wherein oxidizing the dehydrogenation catalyst and the selective hydrogen combustion catalyst comprises exposing the dehydrogenation catalyst and the selective hydrogen combustion catalyst to air.

Aspect 4. The method of aspect 1, wherein no external steam is injected.

Aspect 5. The method of aspect 1, wherein the dehydrogenation catalyst and the selective hydrogen combustion catalyst are arranged in a fixed bed.

Aspect 6. The method of aspect 5, wherein the fixed bed comprises:
(i) sequentially arranged alternating catalyst beds separately comprising the dehydrogenation catalyst and the selective hydrogen combustion catalyst, wherein the alternating catalyst beds are separated by a physical distance;
(ii) a composite bed wherein the dehydrogenation catalyst and the selective hydrogen combustion catalyst are physically in contact in the composite bed; or
(iii) stacked alternating catalyst beds comprising the dehydrogenation catalyst and the selective hydrogen combustion catalyst, wherein the alternating catalyst beds are in physical contact with one or more adjacent catalyst beds.

Aspect 7. The method of aspect 6, wherein the fixed bed comprises (i) and wherein the feedstock is subjected to steam purges between the alternating catalyst beds.

Aspect 8. The method of aspect 6, wherein the fixed bed comprises (ii) and wherein the dehydrogenation catalyst and the selective hydrogen combustion catalyst are present in the form of admixed pellets, particles, or a monolith.

Aspect 9. The method of aspect 6, wherein the fixed bed comprises (ii) and wherein the dehydrogenation catalyst is present as a shell around a selective hydrogen combustion catalyst core.

Aspect 10. The method of aspect 6, wherein the fixed bed comprises (i) or (iii) and wherein the fixed bed comprises from 2 to 20 alternating catalyst beds.

Aspect 11. The method of aspect 6, wherein the fixed bed comprises one or more inert support beds, wherein the one or more inert support beds are configured to ensure even heating of the alkyl benzene feedstock.

Aspect 12. The method of aspect 1, wherein the dehydrogenation catalyst comprises KFeO₂.

Aspect 13. The method of aspect 12, wherein the dehydrogenation catalyst further comprises a promoter comprising a transition metal cation, an alkaline earth metal, or a lanthanide cation, or any combination thereof in an amount of from about 0% to about 10% by weight.

Aspect 14. The method of aspect 13, wherein the promoter comprises Ti, Ga, Ce, Mg, Mo, W, Sn, or any combination thereof.

Aspect 15. The method of aspect 13, wherein the promoter comprises TiO₂, Ga₂O₃, CeO₂, MgO, or any combination thereof.

Aspect 16. The method of aspect 13, wherein the dehydrogenation catalyst comprises Ga₂O₃:TiO₂:CeO₂:MgO:KFeO₂ in a ratio of 2:2:2:1:13.

Aspect 17. The method of aspect 1, wherein the selective hydrogen combustion catalyst comprises LaSrFeO₃, Mg₆MnO₈, (Fe_{0.2}Mn_{0.8})₂O₃, LaCaMnO₃, CaMnTiFeO₃, or any combination thereof.

Aspect 18. The method of aspect 17, wherein the selective hydrogen combustion catalyst further comprises a promoter.

Aspect 19. The method of aspect 18, wherein the promoter comprises an oxide or salt of Li, Na, or K, or any combination thereof.

Aspect 20. The method of aspect 19, wherein the salt comprises a molybdate salt, a vanadate salt, a tungstate salt, or any combination thereof.

Aspect 21. The method of aspect 1, wherein the alkyl benzene feedstock comprises one or more side chains, wherein at least one side chain comprises a carbon-carbon bond.

Aspect 22. The method of aspect 21, wherein the alkyl benzene feedstock comprises ethylbenzene, 4-ethyltoluene, cumene, diethylbenzene, 4-tertbutylethylbenzene, 1,3-diisopropylbenzene, or any combination thereof.

Aspect 23. The method of aspect 1, wherein the alkyl benzene feedstock comprises a gaseous feedstock comprising from about 1 mol % to about 12 mol % of at least one alkyl benzene.

Aspect 24. The method of aspect 23, wherein the gaseous feedstock comprises from about 0 g to about 5 g steam per gram of feedstock.

Aspect 25. The method of aspect 1, wherein the method is performed at a temperature of from about 500° C. to about 700° C.

Aspect 26. A system for converting at least one alkyl benzene feedstock to an alkenyl benzene, the system comprising:
(a) a fixed bed for containing a dehydrogenation catalyst and a selective hydrogen combustion catalyst;
(b) a means for flowing an alkyl benzene feedstock over the fixed bed; and
(c) a means for flowing air over the fixed bed.

Aspect 27. The system of aspect 26, wherein the fixed bed comprises:
(i) sequentially arranged alternating catalyst beds separately comprising the dehydrogenation catalyst and the selective hydrogen combustion catalyst, wherein the alternating catalyst beds are separated by a physical distance;
(ii) a composite bed wherein the dehydrogenation catalyst and the selective hydrogen combustion catalyst are physically in contact in the composite bed; or
(iii) stacked alternating catalyst beds comprising the dehydrogenation catalyst and the selective hydrogen combustion catalyst, wherein the alternating catalyst beds are in physical contact with one or more adjacent catalyst beds.

Aspect 28. The system of aspect 27, wherein the fixed bed comprises (i) and wherein the system further comprises a means for purging the feedstock with steam as the feedstock passes between the alternating catalyst beds.

Aspect 29. The system of aspect 27, wherein the fixed bed comprises (ii) and wherein the dehydrogenation catalyst and the selective hydrogen combustion catalyst are present in the form of admixed pellets, particles, or a monolith.

Aspect 30. The system of aspect 27, wherein the fixed bed comprises (ii) and wherein the dehydrogenation catalyst is present as a shell around a selective hydrogen combustion catalyst core.

Aspect 31. The system of aspect 27, wherein the fixed bed comprises (i) or (iii) and wherein the fixed bed comprises from 2 to 20 alternating catalyst beds.

Aspect 32. The system of aspect 27, wherein the fixed bed comprises one or more inert support beds, wherein the one or more inert support beds are configured to take up sensible heat from an exothermic dehydrogenation reaction ensure even heating of the alkyl benzene and the air.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Synthetic Methods

This section describes the synthesis of the CL-ODH dehydrogenation (DH) catalyst phase. Dehydrogenation catalysts can be prepared via multiple processes including sol-gel synthesis (modified Pechini method), dry ball milling solid state synthesis (e.g. ball milling and palletization), mixing or ball milling in a slurry and extruding, spray drying, granulation, freeze granulation, coprecipitation, precipitation, and/or impregnation.

For one sol-gel synthesis, stoichiometric ratios of metal nitrate precursors (primarily K and Fe, with other transition metal or lanthanide cation promoters such as Ti, Ga, Ce, Mg, Mo, W, K, Cs, Na, Li) were dissolved in deionized water while being stirred at a range between 10-50° C. Citric acid was added to achieve between 1:1 and 5:1 mol citric acid:mol metal cation. Ethylene glycol was added to achieve between 1:1 and 5:1 mol ethylene glycol:mol metal cation and then the solution was heated to 30-95° C. The solution was maintained at 30-95° C. with constant stirring until formation of a gel was observed. The gel was then transferred to an oven for drying at 100-150° C. for >6-12 hrs. The dried gel was calcined in a furnace at 500-1000° C. for 1-12 hrs with a 1-5° C./min ramping rate for heating and cooling. The calcined sample was then pelletized (10-30 MPa), crushed and sieved into 20-80 mesh (250-850 μm).

For one ball milling/slurry synthesis, stoichiometric ratios of metal oxide precursors were weighed and added into the milling jar with a 3-9:1 weight ratio of 1-5 mm milling balls to precursors. The milling balls and precursors were then submerged in ethanol and milled for 1-5 hours. The resulting slurry was dried in an oven at 100-150° C. for 1-12 hrs. The milling balls were sieved from the powder, which was transferred to a crucible and calcined in a furnace at 500-1000° C. for 1-12 hrs with a 1-5° C./min ramping rate for heating and cooling. The calcined sample was then pelletized (10-30 MPa), crushed and sieved into 20-80 mesh (250-850 μm).

This section describes the synthesis of the CL-ODH selective hydrogen combustion (SHC) catalyst phase. SHC catalysts were prepared via ball milling by promoting a mixed metal oxide with lithium, sodium or potassium vanadate, tungstate, niobate or molybdate salts. The salt promoter serves as an oxygen transfer medium for selective hydrogen combustion from the metal oxide. Without the salt promoter, the metal oxides tested exhibit high selectivity toward combustion of the desired carbonaceous products.

For ball milling synthesis, stoichiometric ratios of metal oxide precursors (nitrate, carbonate and/or single-metal oxides are used) were weighed along with the salt promoter and added into the milling jar with a 3-9:1 weight ratio of 1-5 mm milling balls to precursors. Promoters are added to a range of 1-25 wt % of the metal oxide precursor amount, or more preferably 5-20 wt %, or most preferably 1-15 wt %. The milling balls and precursors were then submerged in ethanol and milled for 1-5 hours. The resulting slurry was dried in an oven at 100-150° C. for 1-12 hrs. The milling balls were sieved from the powder, which was transferred to a crucible and calcined in a furnace at 500-1000° C. for 1-12 hrs with a 1-5° C./min ramping rate for heating and cooling. The calcined sample was then pelletized (10-30 MPa), crushed and sieved into 20-80 mesh (250-850 μm).

### Example 2: Reaction Scheme

This section describes the reaction scheme of ethylbenzene CL-ODH with a cyclic redox scheme using mixed metal oxide as a redox catalyst. The two catalyst phases (DH and SHC) are loaded either in a sequence of fixed beds, or in a single mixed composite fixed bed in a stainless-steel U-tube reactor. The sequential bed is arranged such that the reactants flow over the DH catalyst first and then the SHC catalyst. A number of sequential beds between 2 and 10 may be employed to increase conversion. The mixed composite bed is made by a physical mixture of the pellets of each catalyst.

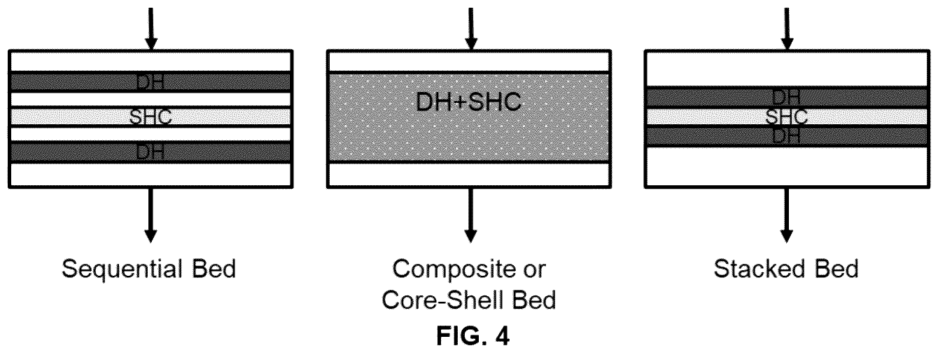

In the first reaction step, 1-12 mol % of alkyl benzene, or more preferably 2-5 mol % alkyl benzene, or most preferably 1-4 mol % alkyl benzene and steam in 0-5 g steam/g feed, or more preferably 0.5-1.0 g steam/g feed, or most preferably 0.0-1.0 g steam/g feed are contacted with the redox catalyst at the conditions discussed above in Section 1. The resulting products are the desired alkenyl benzene, other aromatics and small amounts of CO₂. The redox catalyst is reduced in this step by the hydrogen byproduct. In a second step, air is used to re-oxidize the redox catalyst. Redox cycles are conducted by repeating these two steps (FIG. 4).

The reaction products are analyzed using gas chromatography and mass spectrometry to quantify catalyst performance metrics, namely ethylbenzene conversion, styrene selectivity and selective hydrogen combustion. Owing to the novel sequence of catalytic dehydrogenation followed by selective hydrogen combustion, conversions well above industrial performance are achieved.

This system invites itself for several possible reactor configurations. In one example, ethylbenzene/steam mixture and air are sequentially sent through a fixed bed in a radial direction with steam purges between reductions and oxidation steam. In another example a radial fixed bed is rotated with various zones of the bed rotating through radial flows of ethylbenzene, steam purge, and air.

In yet another example the fixed bed consists of alternating sequences of DH and SHC catalyst. In another embodiment the SHC and DH materials are physically mixed, and in yet another they are composted together. In a further embodiment, the DH catalyst in a composite forms a shell around the SHC material.

In some examples, the steam generated by the SHC reaction combined with the periodic regeneration of the catalyst bed in air are sufficient to suppress coke formation without the addition of steam to the alkenyl benzene. In some examples the high conversion and exothermic nature of the reactor will allow for the export of energy from the reaction.

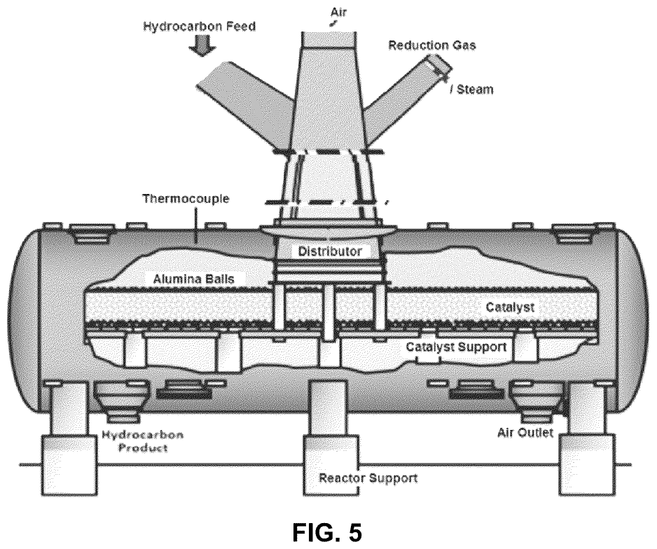

In some examples the sequential, mixed, and or composite beds are placed in a Catofin type reactor configuration (FIG. 5). In these embodiments the flow of the reactant and product gasses vertically through the through the shallow beds undergoes heat exchange with the catalyst bed and inert “support beds” providing adequate preheating to the gaseous feed streams to permit autothermal operations.

### Example 3: Results

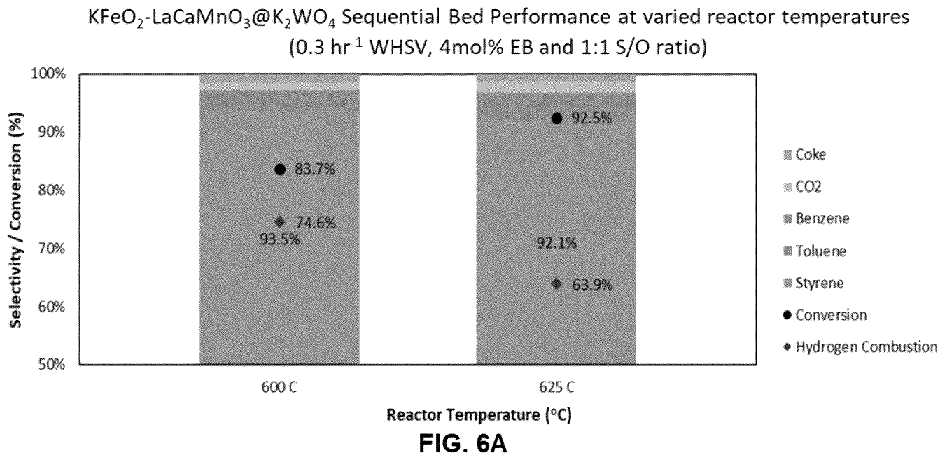

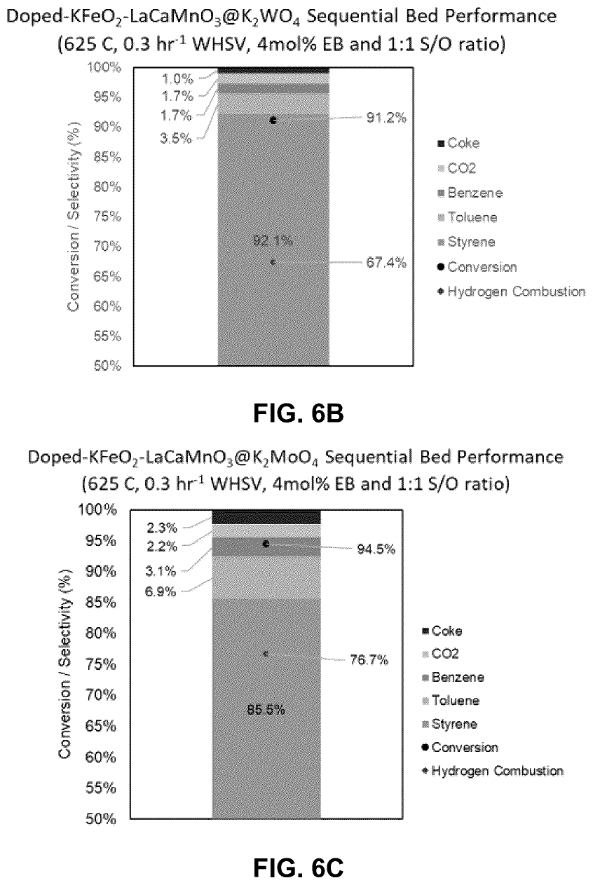

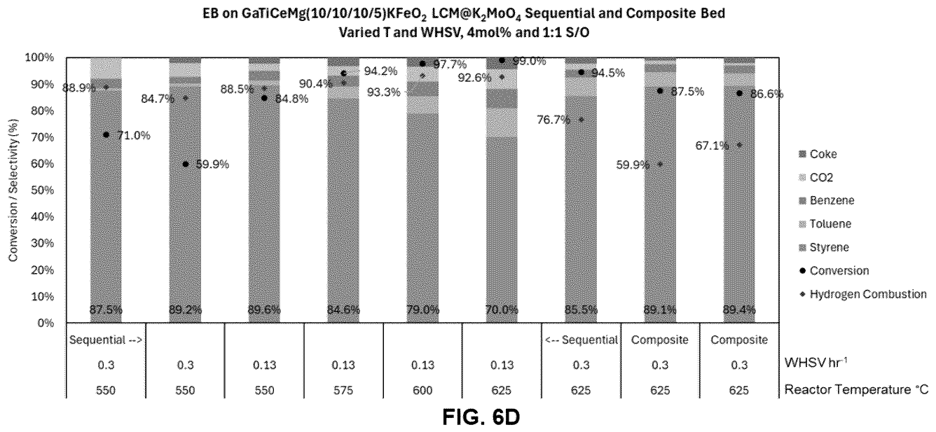

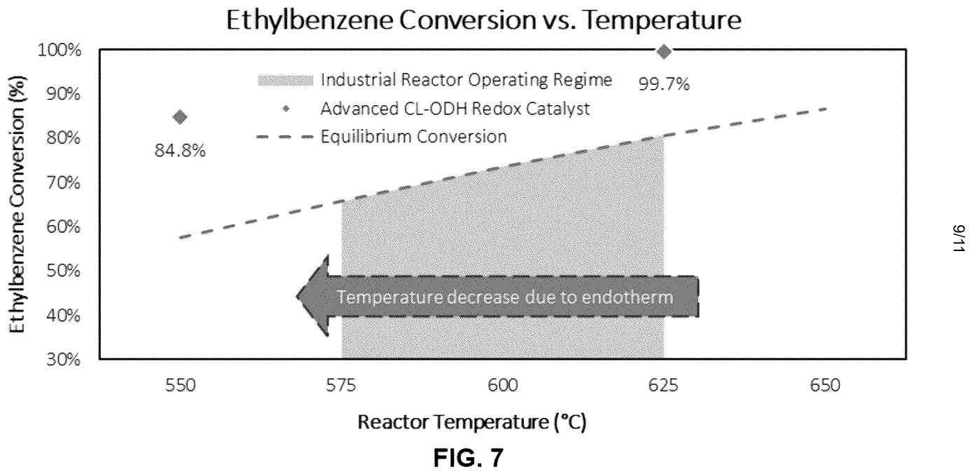

Several compositions of DH and SHC catalysts were tested, with results shown in FIGS. 6A-6D. The equilibrium conversion of the industrial process is shown as a function of temperature in FIG. 7. As can be seen, the equilibrium conversion at 550° C. is 57.7%. The industrial reactor is operated near-adiabatically, which results in a large temperature decline across the catalyst bed due to the endothermic nature of the reaction. The large endothermic temperature differential limits the conversion of alkyl benzenes to alkenyl benzenes. An autothermal self-heating reactor would therefore exhibit higher conversion of the alkyl benzene as the exothermic in-situ hydrogen combustion can offset the endothermic dehydrogenation reaction. In order to demonstrate the novel benefit of SHC and its ability to subvert the equilibrium limitation, the reactor temperature was lowered to 550° C. where an exceptional conversion of 84.8% was unexpectedly achieved (FIG. 6D). Alkyl benzenes conversions up to 99.0% were achieved at 625° C. and 0.13 hr¹ WHSV.

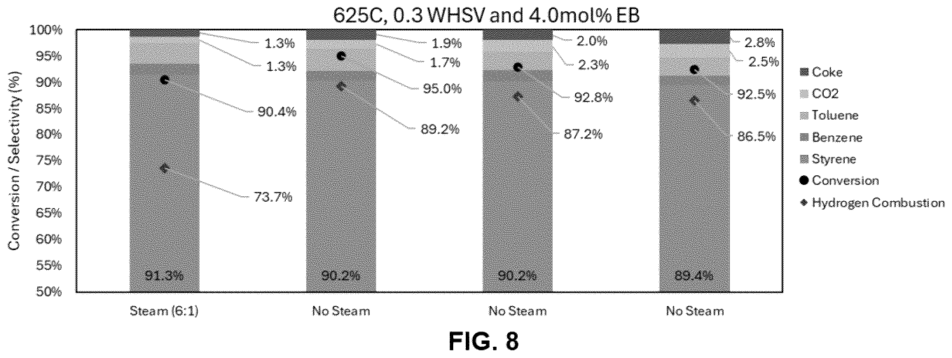

A composite bed of DH and SHC catalyst pellets was also tested with results shown in FIG. 8. The composite bed was formed by physically mixing a weight ratio of catalyst pellets and loading into the reactor in a single catalyst bed.

Tailoring the mixed oxide to enable favorable heat of reaction for the ODH step: Differential scanning calorimetry (DSC) was used to determine the heat of reaction for the selective hydrogen combustion across a range of temperatures (400-800° C.). Autothermal or exothermic operation would be desired to progress the endothermic dehydrogenation reaction to a further extent. Although oxidation of hydrogen is exothermic, the release of oxygen from the selected metal oxide could be endothermic. In most cases the overall reaction heat is positive (endothermic). For instance, many metal oxides such as FeO, Fe₃O₄, and Mn₃O₄ require thermal energy inputs when reacting with H₂. On the other hand, some metal oxides such as CuO can release heat when reacting with hydrogen in theory, but they often undergo spontaneous decomposition, leaving a reduced form of oxide which is much more energy intensive to reduce. This is a key dilemma since an oxide with higher oxygen chemical potential tends to decompose before even contacting with hydrogen in the ODH step, whereas the reduced oxide would be too endothermic when reacting with hydrogen. To make the matter worse, the endothermic alkyl benzene dehydrogenation reaction would require further heat inputs. Although such heat can be provided by the regeneration step in CL-ODH, this indirect heating is not effective. Moreover, the large temperature drops during the ODH step itself would greatly limit the production yield.

DSC data shown below (Table 1) demonstrate the range of possible heat release from different materials with and without a promoter. The data are expressed as a ratio of the oxidation to reduction heat release ratio in order to show the tunable nature of the materials. It is noted that, in several cases, promoted oxide showed significantly larger exotherm. This is rather unexpected since adding the promoter actually make the oxides more difficult to reduce. One would have expected that the more difficult to reduce oxide should require more reaction energy inputs, but this is not the case. Particularly, promoted Fe/Mn mixed oxides showed significantly higher exotherm than its unpromoted counterparts. In fact, the reason for such an enhancement is precisely because the presence of the promoter kinetically limits the lattice oxygen release (i.e. spontaneous decomposition in the case of, e.g. CuO) but did not change the thermodynamic potential of these reactive oxygen species. As a result, one can expect an overall exothermic reaction for the ODH step (i.e. reduction+dehydrogenation reaction will be net exothermic). Table 2 shows the results of a hydrogen-ethylene cofeed temperature-programmed reduction (H₂-C₂H₄-TPR) in order to determine the relative selectivity of each oxide to hydrogen combustion versus carbonaceous species. Here, it can clearly be seen that the promoted oxides are highly selective towards hydrogen combustion in the presence of hydrocarbons, making them ideal oxides for the proposed CL-ODH reactions. It is non-obvious that the exothermicity increases when adding a promoter. By adjusting the promoter amount and type, the disclosed catalyst materials have controllable exothermicity. This enables preservation of metal oxides in a high-oxidation state, unexpectedly and excellently inhibiting unselective oxidation of carbonaceous compounds.

TABLE 1
TGA-DSC Data for Heat Release for Tested Catalysts

| | Reduction Peak | Reduction [symbol] | Oxidation Peak | Reduction:Oxidation |
|---|---|---|---|---|
| | (kJ/mol [symbol] ) | ΔH [symbol] (kJ/mol) | (kJ/mol [symbol] ) | Heat Release Ratio |
| LaSrFeO₃ | −88.8 | 36.1 | −112.0 | 0.7 [symbol] |
| LaSrFeO₃ promoted | −19.5 | 105.4 | −152.5 | 0.13 |
| Mg [symbol] MnO [symbol] | −47.9 | 77.0 | −87.0 | 0.55 |
| Mg [symbol] MnO [symbol] promoted | −74.4 | 50.5 | −100.6 | 0.74 |
| (Fe_{0.2}Mn [symbol] )₂O₃ | −36.1 | 88.8 | −122.8 | 0.29 |
| (Fe_{0.2}Mn [symbol] )₂O₃ promoted | −104.4 | 20.5 | −71.6 | 1.46 |
| LaCaMnO [symbol] | −128.1 | −3.2 | −95.5 | 1.34 |
| LaCaMnO [symbol] promoted | −108.2 | 16.7 | −100.5 | 1.08 |
| CaMnTiFeO₃ | −46.3 | 78. [symbol] | −155.5 | 0.30 |
| CaMnTiFeO₃ promoted | −39.8 | 85.1 | −148.3 | 0.27 |

[symbol]

indicates data missing or illegible when filed

TABLE 2
H₂—C₂H₄-TPR Cofeed Experimental Results

| | Combustion Onset | Peak Reduction | Hydrogen | Ethylene | Hydrogen Combustion |
|---|---|---|---|---|---|
| | Temperature (° C.) | Temperature (° C.) | Combusted (mmol) | Combusted (mmol) | Selectivity (%) |
| Mg [symbol] MnO₈ | 475 | 580 | 1.2 | 0.9 | 57% |
| Mg [symbol] MnO₈ promoted | 590 | 724 | 0.4 | 0.0 | 99% |
| (Fe_{0.2}Mn_{0.8})₂O₃ | 385 | 592 | 3.2 | 1.6 | 66% |
| (Fe_{0.2}Mn_{0.8})₂O₃ promoted | 543 | 750 | 1.5 | 0.0 | 100% |
| LaCaMnO₃ | 363 | 635 | 1.2 | 0.0 | 100% |
| LaCaMnO₃ promoted | 380 | 623 | 1.0 | 0.0 | 100% |
| CaMnTiFeO₃ | 389 | 655 | 1.0 | 0.1 | 88% |
| CaMnTiFeO₃ promoted | 513 | 669 | 0.9 | 0.0 | 100% |

[symbol]

indicates data missing or illegible when filed

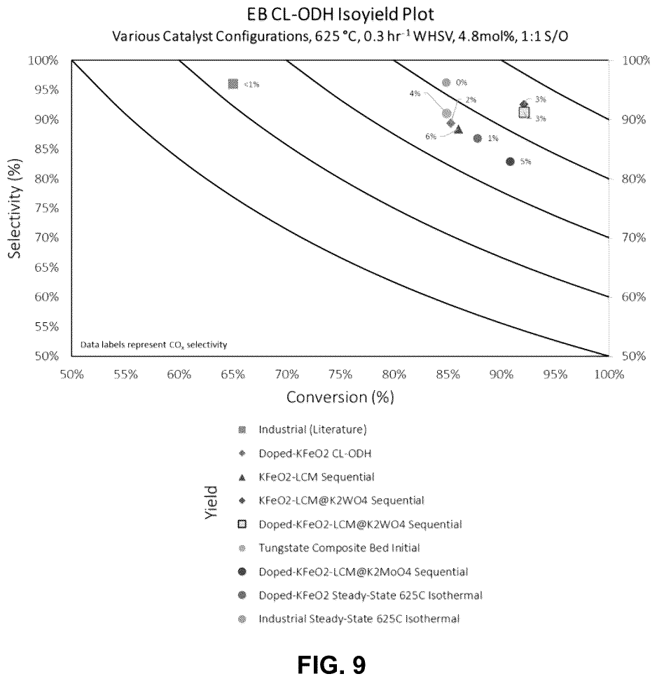

FIG. 9 summarizes the best overall styrene yields obtained with ethylbenzene CL-ODH using a variety of multifunctional redox catalysts.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

### REFERENCES

1. Cavani, F.; et al. Alternative Processes for the Production of Styrene. Appl. Catal. Gen. 1995, 133 (2), 219-239. DOI: 10.1016/0926-860X(95)00218-9.
2. Lee, E. H. Iron Oxide Catalysts for Dehydrogenation of Ethylbenzene in the Presence of Steam. Catal. Rev. 1974, 8 (1), 285-305. DOI: 10.1080/01614947408071864.
3. Nawaz, Z. Dynamic Reactor Modeling Of Catofin® Fixed-Bed Iso-Butane Dehydrogenation Reactor Using ACM; 2015.

What is claimed is:

1. A method for converting at least one alkyl benzene feedstock to an alkenyl benzene, the method comprising:
(a) contacting the at least one alkyl benzene feedstock with a dehydrogenation catalyst and a selective hydrogen combustion catalyst to produce the alkenyl benzene and selectively combust a hydrogen byproduct, wherein the selective hydrogen combustion catalyst releases at least 30 kJ of thermal energy per mole of hydrogen combusted into steam;
(b) oxidizing the dehydrogenation catalyst and the selective hydrogen combustion catalyst; and
repeating steps (a) and (b) one or more additional times.

2. The method of claim 1, wherein a combination of step (a) and step (b) has a heat of reaction of less than 100 kJ/mol alkyl benzene converted.

3. The method of claim 1, wherein oxidizing the dehydrogenation catalyst and the selective hydrogen combustion catalyst comprises exposing the dehydrogenation catalyst and the selective hydrogen combustion catalyst to air.

4. The method of claim 1, wherein no external steam is injected.

5. The method of claim 1, wherein the dehydrogenation catalyst comprises KFeO₂.

6. The method of claim 5, wherein the dehydrogenation catalyst further comprises a promoter comprising a transition metal cation, an alkaline earth metal, or a lanthanide cation, or any combination thereof in an amount of from about 0% to about 10% by weight.

7. The method of claim 6, wherein the promoter comprises Ti, Ga, Ce, Mg, Mo, W, Sn, or any combination thereof.

8. The method of claim 1, wherein the selective hydrogen combustion catalyst comprises LaSrFeO₃, Mg₆MnO₈, (Fe_{0.2}Mn_{0.8})₂O₃, LaCaMnO₃, CaMnTiFeO₃, or any combination thereof.

9. The method of claim 8, wherein the selective hydrogen combustion catalyst further comprises a promoter, wherein the promoter comprises an oxide or salt of Li, Na, or K, or any combination thereof.

10. The method of claim 1, wherein the alkyl benzene feedstock comprises one or more side chains, wherein at least one side chain comprises a carbon-carbon bond.

11. The method of claim 10, wherein the alkyl benzene feedstock comprises ethylbenzene, 4-ethyltoluene, cumene, diethylbenzene, 4-tertbutylethylbenzene, 1,3-diisopropylbenzene, or any combination thereof.

12. The method of claim 1, wherein the alkyl benzene feedstock comprises a gaseous feedstock comprising from about 1 mol % to about 12 mol % of at least one alkyl benzene.

13. The method of claim 12, wherein the gaseous feedstock comprises from about 0 g to about 5 g steam per gram of feedstock.

14. A system for converting at least one alkyl benzene feedstock to an alkenyl benzene, the system comprising:
(a) a fixed bed for containing a dehydrogenation catalyst and a selective hydrogen combustion catalyst;
(b) a means for flowing an alkyl benzene feedstock over the fixed bed; and
(c) a means for flowing air over the fixed bed.

15. The system of claim 14, wherein the fixed bed comprises:
(i) sequentially arranged alternating catalyst beds separately comprising the dehydrogenation catalyst and the selective hydrogen combustion catalyst, wherein the alternating catalyst beds are separated by a physical distance;
(ii) a composite bed wherein the dehydrogenation catalyst and the selective hydrogen combustion catalyst are physically in contact in the composite bed; or
(iii) stacked alternating catalyst beds comprising the dehydrogenation catalyst and the selective hydrogen combustion catalyst, wherein the alternating catalyst beds are in physical contact with one or more adjacent catalyst beds.

16. The system of claim 15, wherein the fixed bed comprises (i) and wherein the system further comprises a means for purging the feedstock with steam as the feedstock passes between the alternating catalyst beds.

17. The system of claim 15, wherein the fixed bed comprises (ii) and wherein the dehydrogenation catalyst and the selective hydrogen combustion catalyst are present in the form of admixed pellets, particles, or a monolith.

18. The system of claim 15, wherein the fixed bed comprises (ii) and wherein the dehydrogenation catalyst is present as a shell around a selective hydrogen combustion catalyst core.

19. The system of claim 15, wherein the fixed bed comprises (i) or (iii) and wherein the fixed bed comprises from 2 to 20 alternating catalyst beds.

20. The system of claim 15, wherein the fixed bed comprises one or more inert support beds, wherein the one or more inert support beds are configured to take up sensible heat from an exothermic dehydrogenation reaction ensure even heating of the alkyl benzene and the air.
